(19) United States
(12) Patent Application Publication
(10) Pub. No.: US 2017/0065156 A1
(43) Pub. Date: Mar. 9, 2017

(54) SCANNING-TYPE ENDOSCOPE

(71) Applicant: OLYMPUS CORPORATION, Tokyo (JP)

(72) Inventors: Daiki ARIYOSHI, Tokyo (JP); Yuji SAKAI, Tokyo (JP); Soichiro KOSHIKA, Tokyo (JP); Hiroyuki TAKIZAWA, Tokyo (JP)

(73) Assignee: OLYMPUS CORPORATION, Tokyo (JP)

(21) Appl. No.: 15/353,811

(22) Filed: Nov. 17, 2016

Related U.S. Application Data
(63) Continuation of application No. PCT/JP2015/073866, filed on Aug. 25, 2015.

(30) Foreign Application Priority Data
Dec. 12, 2014 (JP) 2014-252107

(51) Int. Cl. A61B 1/00; G02B 26/10; G02B 23/24

(52) U.S. Cl. CPC A61B 1/00165; A61B 1/00172; G02B 23/2469; G02B 26/103; G02B 23/2476

(57) ABSTRACT

A scanning-type endoscope includes: a scanning portion including a light guiding portion and an actuator configured to oscillate a distal end of the light guiding portion; a cylindrical member provided along the light guiding portion; and a holding portion provided on the cylindrical member and configured to hold the scanning portion at a predetermined position on a plane perpendicular to a longitudinal direction of the cylindrical member and configured so that the scanning portion is slidable in the longitudinal direction.

### CROSS REFERENCE TO RELATED APPLICATION

This application is a continuation application of PCT/JP2015/073866 filed on Aug. 25, 2015 and claims benefit of Japanese Application No. 2014-252107 filed in Japan on Dec. 12, 2014, the entire contents of which are incorporated herein by this reference.

### BACKGROUND OF THE INVENTION

1. Field of the Invention

The present invention relates to a scanning-type endoscope which causes illuminating light to be emitted, detects return light, and images the return light while performing scanning with an illumination fiber.

2. Description of the Related Art

An electronic endoscope is known which photoelectrically converts an object image to display an image of the object on a monitor by an image pickup apparatus having a solid image pickup device such as a CCD and a CMOS. Further, an optical scanning-type endoscope apparatus is known as an apparatus which displays an image of an object without the solid image pickup device technique.

For example, Japanese Patent Application Laid-Open Publication No. 2012-231911 discloses an optical scanning apparatus and a scanning-type observation apparatus which correct influence by aberration of an optical system to reduce variation in resolution capability. The optical scanning apparatus has an insertion portion in an elongated shape, and the insertion portion is provided with an illumination fiber configured to guide laser light to a distal end portion.

A distal end of the illumination fiber is adapted to be spirally displaced from a center outward in a radius direction by gradually increasing an amplitude of a drive signal given to a piezoelectric device.

### SUMMARY OF THE INVENTION

A scanning-type endoscope of an aspect of the present invention includes: a scanning portion including a light guiding portion configured to guide illuminating light emitted from a light source portion and emit the illuminating light from a distal end, and an actuator configured to oscillate the distal end of the light guiding portion in order to perform scanning on an observation target with the illuminating light; a cylindrical member including a space containing the scanning portion and provided along the light guiding portion; and a holding portion provided on the cylindrical member and configured to hold the scanning portion at a predetermined position on a plane perpendicular to a longitudinal direction of the cylindrical member and configured so that the scanning portion is slidable in the longitudinal direction.

### BRIEF DESCRIPTION OF THE DRAWINGS

FIG. 1 is a diagram illustrating a scanning-type endoscope apparatus;

FIG. 2 is a diagram illustrating a configuration of a distal end portion of an insertion portion of a scanning-type endoscope and an optical scanning unit;

FIG. 3 is a cross-sectional view of an arrow Y3-Y3 line in FIG. 2;

FIG. 4A is a diagram illustrating a relationship between a holding body and a ferrule;

FIG. 4B is a cross-sectional view of an arrow Y4B-Y4B line in FIG. 4A;

FIG. 5A is a diagram illustrating a ferrule which does not rotate in a circumferential direction relative to the holding body;

FIG. 5B is a cross-sectional view of an arrow Y5B-Y5B line in FIG. 5A;

FIG. 6A is a diagram illustrating a ferrule which does not move in an axial direction relative to the holding body;

FIG. 6B is a cross-sectional view of an arrow Y6B-Y6B line in FIG. 6A;

FIG. 7A is a diagram illustrating a ferrule which neither rotates in the circumferential direction nor moves in the axial direction relative to the holding body; and

FIG. 7B is a cross-sectional view of an arrow Y7B-Y7B line in FIG. 7A.

### DETAILED DESCRIPTION OF THE PREFERRED EMBODIMENT(S)

An embodiment of the present invention will be described below with reference to drawings.

Note that each drawing used in the description below is a schematic drawing, and, as for a dimensional relationship, reduced scale and the like of each member, the reduced scale differs for each component so that each of the components is recognizably shown on the drawing. The present invention is not limited only to the number of components, shapes of the components, a ratio of sizes of the components, a relative positional relationship among the respective components shown in the drawings.

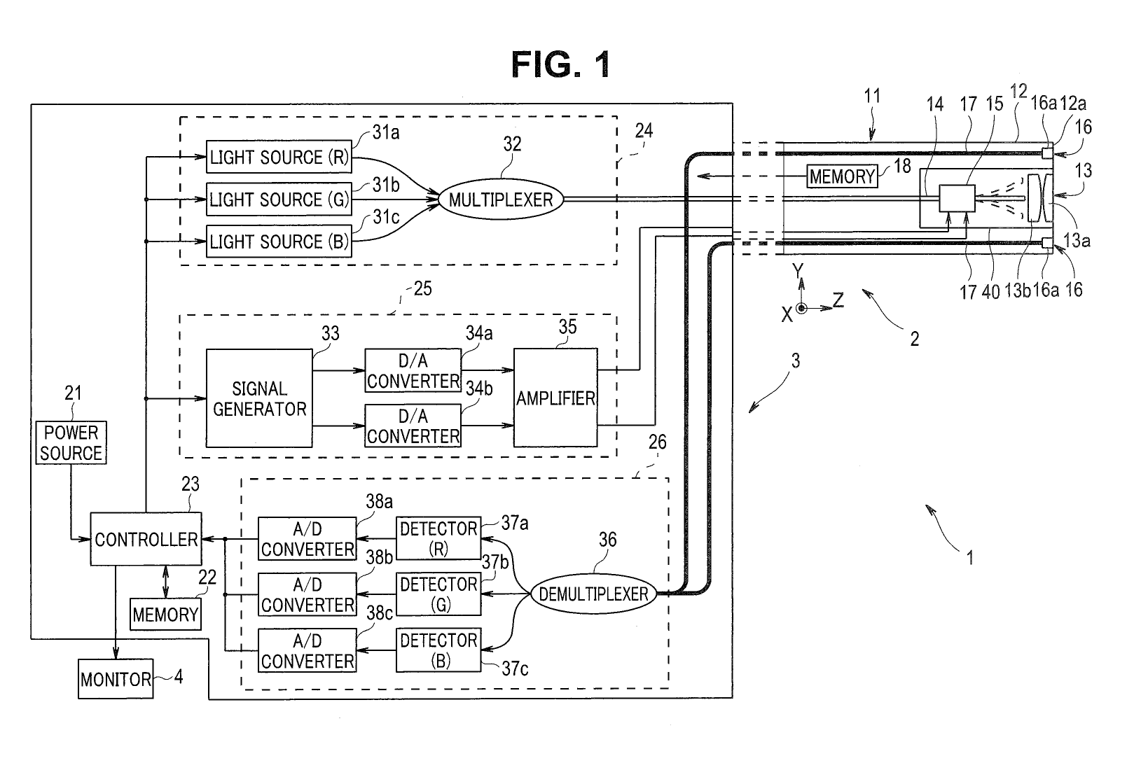

As shown in FIG. 1, a scanning-type endoscope apparatus 1 is configured having a scanning-type endoscope (hereinafter simply referred to as an endoscope) 2, a body apparatus 3 to which the endoscope 2 is connected, and a monitor 4.

The endoscope 2 emits illuminating light to a subject while performing scanning with the illuminating light and obtains return light from the subject. A subject image generated by the body apparatus 3 is displayed on the monitor 4.

The endoscope 2 has an elongated insertion portion 11 configured to be inserted into a living body. The insertion portion 11 is configured mainly with a tube body having predetermined flexibility, and a distal end portion 12 is provided on a distal end side of the insertion portion 11.

A connector not shown and the like are provided on a proximal end side of the insertion portion 11. The endoscope 2 is configured to be attachable to and detachable from the body apparatus 3 via the connector or the like.

On a distal end face 12a of the distal end portion 12, a distal end illumination lens 13a which is an optical member constituting an illumination optical system 13, and a condensing lens 16a which is an optical member constituting a detection optical system 16 are provided.

Reference numeral 13b denotes a second illumination lens, which is one of optical members constituting the illumination optical system 13. The illumination optical system 13 is configured being provided with one or more optical members. The detection optical system 16 is configured with the condensing lens 16a and a detection fiber 17.

Inside the insertion portion 11, the illumination optical system 13, an illumination fiber 14, an actuator 15 and the detection fiber 17 are provided.

The illumination fiber 14 is a light guiding portion, which guides illuminating light emitted from a light source unit 24 to be a light source portion to be described later. The guided illuminating light passes through the illumination optical system 13 and emitted to an object which is an observation target from a distal end.

The actuator 15 is, for example, a piezoelectric device and is a scanner driving portion provided on a distal end side of the illumination fiber 14. The piezoelectric device is provided with two pairs of electrodes facing each other to be described later at four positions obtained by dividing the piezoelectric device into four in a circumferential direction. The piezoelectric device is driven based on a drive signal from a driver unit 25 to be described later and performs oscillation control of a distal end of the illumination fiber 14 to perform scanning on an observation target with illuminating light along a predetermined trajectory.

The illumination fiber 14 and the actuator 15 constitute an optical scanning unit 40 which is a scanning portion.

The detection fiber 17 is inserted along an inner circumference of the insertion portion 11. The detection fiber 17 transmits return light from the observation target received by the condensing lens 16a to a detection unit 26 to be described later. That is, the condensing lens 16a is arranged on a distal end of the detection fiber 17.

Note that the detection fiber 17 is a fiber bundle of at least two or more fibers. When the endoscope 2 is connected to the body apparatus 3, the detection fiber 17 is connected to a demultiplexer 36 to be described later.

Reference numeral 18 denotes an endoscope memory. Various information about the endoscope 2 is stored in the endoscope memory 18. The endoscope memory 18 is provided inside the insertion portion 11. When the endoscope 2 is connected to the body apparatus 3, the endoscope memory 18 and a controller 23 are connected via a signal line not shown. The various information stored in the endoscope memory 18 is read out by the controller 23.

The body apparatus 3 is provided with a power source 21, a body memory 22, the controller 23, the light source unit 24, the driver unit 25, the detection unit 26, and the like.

The light source unit 24 is configured having three light sources 31a, 31b and 31c and a multiplexer 32.

The driver unit 25 is configured having a signal generator 33, a digital/analog (hereinafter referred to as D/A) converters 34a and 34b, and an amplifier 35.

The detection unit 26 is configured having demultiplexer 36, detectors 37a, 37b and 37c, and analog/digital (hereinafter referred to as A/D) converters 38a, 38b and 38c.

The power source 21 supplies power to the controller 23 in response to an operation of a power switch or the like not shown.

A control program for performing control of the whole body apparatus 3 and the like are stored in the body memory 22.

When power supply from the power source 21 is started, the controller 23 reads out the control program from the body memory 22 and performs control of the light source unit 24, the driver unit 25 and a detection unit 27.

The light sources 31a, 31b and 31c of the light source unit 24 emit lights with different wavelength bands, for example, lights with wavelength bands of R (red), G (green) and B (blue) to the multiplexer 32 based on control of the controller 23. The multiplexer 32 multiplexes the lights with the R, G and B wavelength bands emitted from the light sources 31a, 31b and 31c and emits the multiplexed light to the illumination fiber 14.

The signal generator 33 of the driver unit 25 outputs a drive signal for causing the distal end of the illumination fiber 14 to perform scanning in a desired direction, for example, in an elliptical spiral, based on control of the controller 23.

More specifically, the signal generator 33 outputs a drive signal to cause the distal end of the illumination fiber 14 to be driven in an left-and-right direction (an X axis direction) relative to a longitudinal axis of the insertion portion 11 to the first D/A converter 34a, and outputs a drive signal to cause the distal end of the illumination fiber 14 to be driven in an up-and-down direction (a Y axis direction) relative to an insertion axis of the insertion portion 11 to the second D/A converter 34b.

Note that a longitudinal axis direction of the insertion portion 11 is defined as a Z axis direction, and two directions orthogonal to the Z axis direction and orthogonal to each other are defined as the X axis direction and the Y axis direction.

The D/A converters 34a and 34b convert the respective inputted drive signals from digital signals to analog signals and output the analog signals to the amplifier 35.

The amplifier 35 amplifies the inputted drive signals and outputs the drive signals to the two pairs of electrodes 49a, 49b, 49c and 49d provided on the actuator 15. The actuator 15 as a vibrating portion causes the distal end of the illumination fiber 14, which is a free end, to be oscillated and perform scanning in an elliptical spiral, based on the drive signals outputted from the amplifier 35 to the electrodes 49a, 49b, 49c and 49d.

Thereby, the light emitted from the light source unit 24 to the illumination fiber 14 is sequentially emitted onto a subject in an elliptical spiral.

Return light, the light emitted to the subject and reflected by a surface area of the subject, is guided to the demultiplexer 36 of the detection unit 26 by the detection fiber 17. The demultiplexer 36 is, for example, a dichroic mirror, and the demultiplexer 36 demultiplexes the return light in a predetermined wavelength band.

More specifically, the demultiplexer 36 demultiplexes the return light guided by the detection fiber 17 to return lights of the R, G and B wavelength bands and outputs the return lights of the R, G and B wavelength bands to the detectors 37a, 37b and 37c, respectively.

The detectors 37a, 37b and 37c detect light intensities of the return lights of the R, G and B wavelength bands, respectively. Signals of the light intensities detected by the detectors 37a, 37b and 37c are outputted to the A/D converters 38a, 38b and 38c, respectively. The A/D converters 38a, 38b and 38c convert the signals of the light intensities outputted from the respective detectors 37a, 37b and 37c from analog signals to digital signals and output the digital signals to the controller 23.

The controller 23 performs predetermined image processing for the digital signals from the A/D converters 38a, 38b and 38c to generate an object image, and displays the object image on the monitor 4.

Here, description will be made on a detailed configuration of the optical scanning unit 40 provided inside the distal end portion 12 of the insertion portion 11.

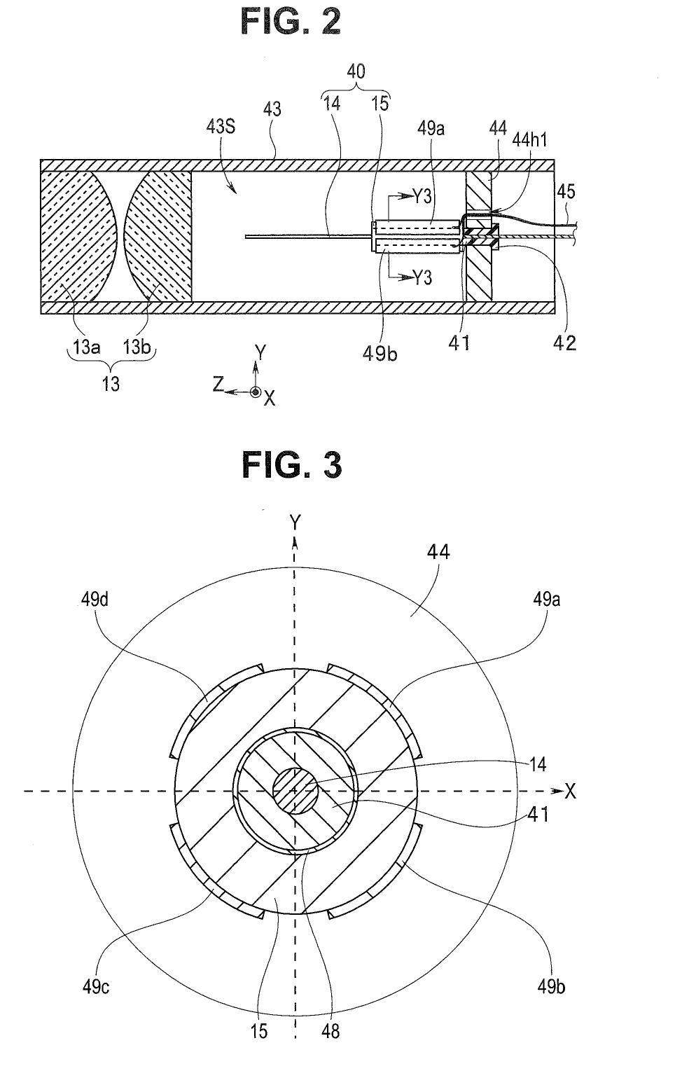

As described above, the optical scanning unit 40 configured with the illumination fiber 14 and the actuator 15 is contained in a space portion 43S provided in a frame body 43, which is a cylindrical member to which the illumination lenses 13a and 13b constituting the distal end portion 12 are integrally fixed, as shown in FIG. 2. The distal end of the illumination fiber 14 is arranged in the space portion 43S as a free end.

As shown in FIGS. 2 and 3, the illumination fiber 14 is insertedly arranged and held in a ferrule 41. The actuator 15 is provided on an outer side face of the ferrule 41.

The ferrule 41 is formed with material, such as zirconia and nickel, which makes it possible to easily perform hole processing corresponding to an outer diameter (for example, 125 μm) of the illumination fiber 14 with high accuracy (for example, +1 μm).

As shown in FIG. 3, the ferrule 41 is, for example, in a cylindrical shape, and a central through-hole based on the diameter of the illumination fiber 14 is formed at a center of a section of the cylindrical shape. The illumination fiber 14 is arranged in the central through-hole in a predetermined state and is integrally fixed to the ferrule 41 with adhesive or the like.

Note that a clearance of the central through-hole is formed small in order to make an adhesive layer as thin as possible, and adhesive with a low viscosity is used.

The actuator 15 in a predetermined pipe shape is arranged on an outer circumferential face of the ferrule 41. An electrode 48 is provided on an inner circumferential face of the actuator 15, and the two pairs of electrodes 49a, 49b, 49c and 49d described above are arranged on an outer circumferential face at predetermined intervals.

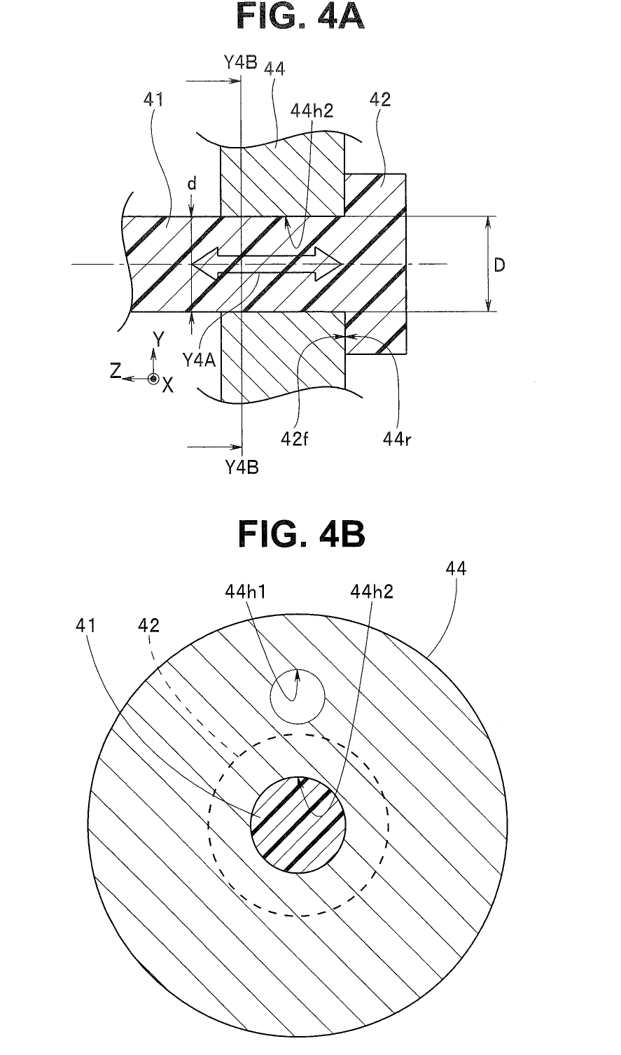

As shown in FIG. 2, a proximal end side of the ferrule 41 is arranged in a holding body 44 which is a holding portion. The holding body 44 is in a disc shape with a predetermined thickness, and an axial-direction first through-hole 44h1 and an axial-direction second through-hole 44h2 (see FIG. 4) are formed in the holding body 44. A plurality of lead wires 45 are insertedly arranged in the axial-direction first through-hole 44h1, and the ferrule 41 is fittedly arranged in the axial-direction second through-hole 44h2.

The axial-direction second through-hole 44h2 is a ferrule mounting hole (hereinafter also referred to as the ferrule mounting hole 44h2). An inner diameter d of the axial-direction second through-hole 44h2 shown in FIG. 4A is set larger than an outer diameter D of the ferrule 41 by a predetermined length. That is, a predetermined clearance is provided between the axial-direction second through-hole 44h2 and the ferrule 41.

As shown in FIGS. 2, 4A and 4B, the ferrule 41 has a diameter-increased portion 42, which is a flange projecting from the outer circumferential face outward with a predetermined height, on a proximal end of the ferrule 41. A distal end side face of the diameter-increased portion 42 is a contact face 42f, which is a plane perpendicular to a longitudinal axis of the ferrule 41.

The ferrule 41 is inserted into the ferrule mounting hole 44h2 from a proximal end face 44r side of the holding body 44. Then, the contact face 42f of the diameter-increased portion 42 comes into contact with the proximal end face 44r of the holding body 44, and the ferrule 41 is in a predetermined arrangement state.

That is, in the present embodiment, the ferrule 41 is not integrally fixed to the holding body 44 by adhesion but is fittedly arranged in the ferrule mounting hole 44h2.

The holding body 44 in which the ferrule 41 is arranged is integrally fixed at a predetermined position on a proximal end side of a frame body 43 by adhesion or the like. In this fixation state, a central axis of the holding body 44 corresponds to a central axis of the frame body 43. Therefore, the frame body 43 in which the illumination optical system 13 is provided is provided along the illumination fiber 14.

Further, the contact face 42f of the diameter-increased portion 42 comes into contact with the proximal end face 44r of the holding body 44, and the distal end of the illumination fiber 14 constituting the optical scanning unit 40 is arranged at a predetermined position in a longitudinal direction of the frame body 43.

Note that the ferrule 41 is not limited to a cylindrical shape but may be in any prism shape such as a quadrangular prism. If the ferrule 41 is in a prism shape, the actuator 15 in a rectangular parallelepiped shape with a predetermined size is arranged at a predetermined position on each plane of the prism.

Further, a resonance frequency for causing the illumination fiber 14 to be largely oscillated is determined by the diameter of the illumination fiber 14 and a length of the free end, which is a projection length from a distal end face of the ferrule 41.

The distal end of the illumination fiber 14 provided in the scanning-type endoscope 2 configured as described above is oscillated by the ferrule 41 being vibrated.

The ferrule 41 is vibrated by the actuator 15 being driven in response to drive signals supplied from the D/A converters 34a and 34b to the electrodes 49a, 49b, 49c and 49d.

Then, by the actuator 15 being drive-controlled, the oscillation of the distal end of the illumination fiber 14 by the vibration of the ferrule 41 is controlled, and scanning is performed on the observation target in an elliptical spiral with illuminating light.

At this time, the ferrule 41 also vibrates in the Z direction as shown by an arrow Y4A in FIG. 4A. However, the ferrule 41 is not integrally fixed to the holding body 44 by adhesion but is fittedly arranged in the ferrule mounting hole 44h2 as described above.

Therefore, the ferrule 41 is slidingly moves in a direction of the arrow Y4A in the ferrule mounting hole 44h2 by the vibration in the Z direction which has occurred in the ferrule 41. Therefore, the Z-direction vibration indicated by the arrow Y4A which has occurred in the ferrule 41 is difficult to be propagated to the holding body 44.

As a result, the Z-direction vibration indicated by the arrow Y4A which has occurred in the ferrule 41 is hardly propagated to the frame body 43 via the holding body 44, and, thereby, it is possible to prevent such a trouble from happening that the frame body 43 shakes, and an observation target image is disturbed.

Note that, when the Z-direction vibration occurs in the frame body 43, the vibration is propagated to the holding body 44 because the holding body 44 is integrally fixed to the frame body 43. However, since the ferrule 41 is fittedly arranged in the ferrule mounting hole 44h2 of the holding body 44 as described above, the vibration propagated to the holding body 44 is difficult to be propagated to the ferrule 41.

As a result, it is possible to prevent it from happening that a trouble occurs in oscillation of the distal end of the illumination fiber 14 by vibration of the ferrule 41 because of vibration from outside being transmitted to the optical scanning unit 40, and elliptical spiral scanning on an observation target with illuminating light is disturbed.

Note that the illumination fiber 14 is omitted in FIG. 4A.

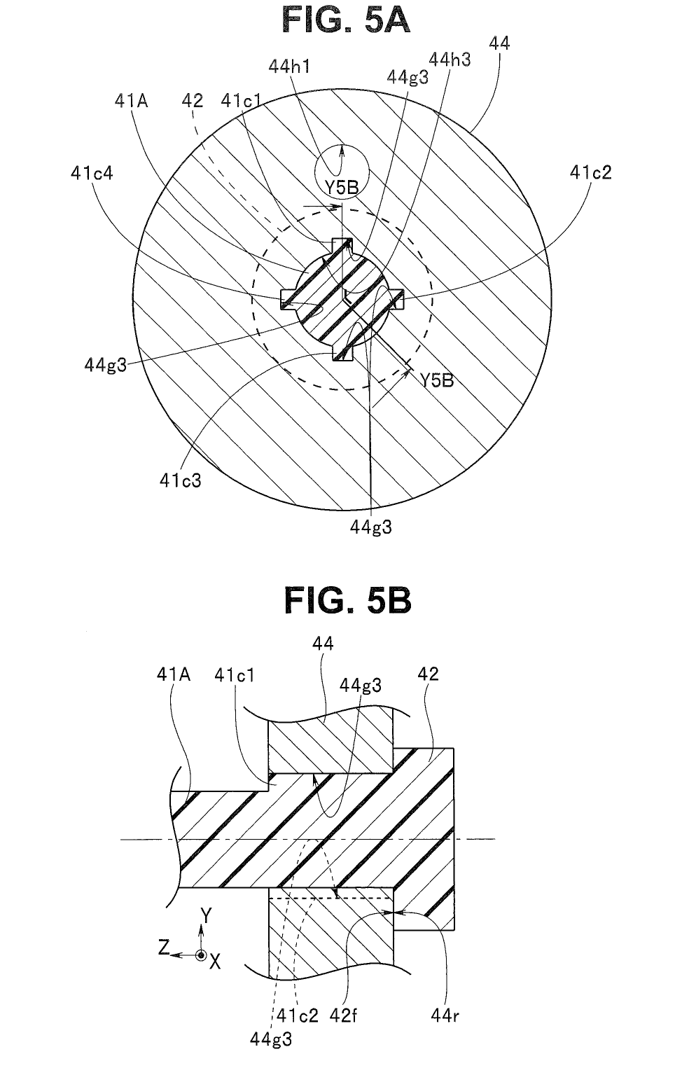

Here, other configuration examples of the ferrule will be described with reference to FIGS. 5A, 5B, 6A, 6B, 7A and 7B.

A first ferrule 41A shown in FIGS. 5A and 5B is provided with a plurality of projecting portions projecting outward in a circumferential direction at intervals. In the present embodiment, projection portions 41c1, 41c2, 41c3 and 41c4, which are the projecting portions, are projected whirl-stops. A ferrule mounting hole 44h3 having recess grooves 44g3 as groove portions where the projection portions 41c1, 41c2, 41c3 and 41c4 are arranged, respectively, is formed in the holding body 44.

The projection portions 41c1, 41c2, 41c3 and 41c4 of the first ferrule 41A are arranged being fitted in the four recess grooves 44g3 formed in the ferrule mounting hole 44h3.

As a result, the operation and effects described above are obtained, and a trouble that the ferrule 41 rotatingly moves in the circumferential direction by vibration is solved. Therefore, a more preferable observation target image can be obtained.

Note that the first ferrule 41A and the holding body 44 may be provided with recess grooves and projection portions, respectively, though it is not shown.

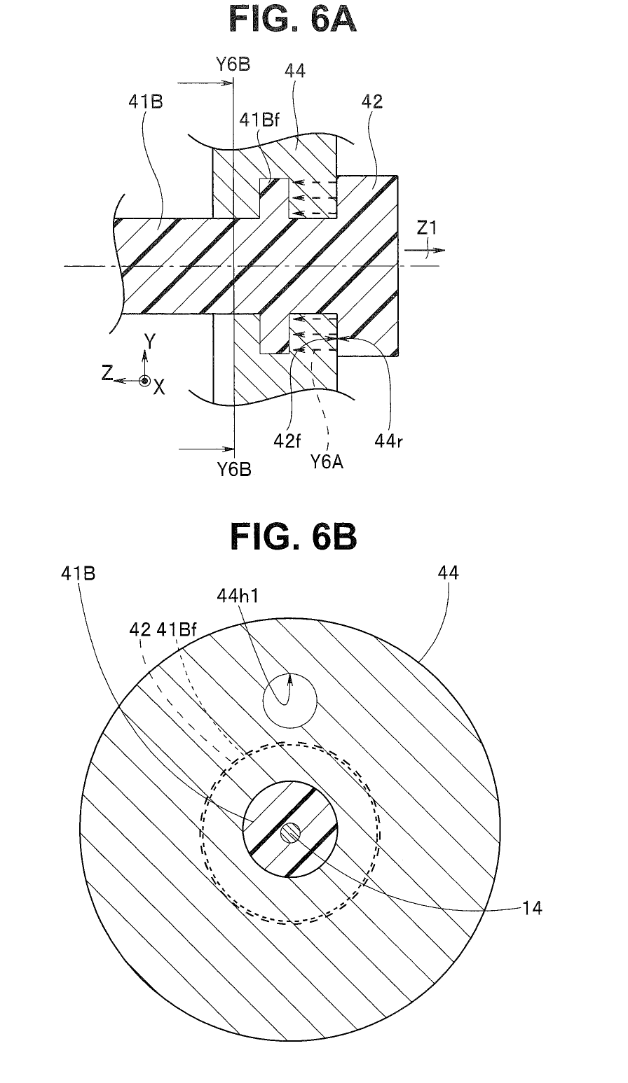

A second ferrule 41B shown in FIGS. 6A and 6B has a flange portion 41Bf as a projecting portion projecting outward from an outer circumferential face of the ferrule 41B at a predetermined position of a midway portion. The flange portion 41Bf is a Z-direction positioning portion of the ferrule 41B and arranged in the holding body 44.

By the flange portion 41Bf of the second ferrule 41B being arranged in the holding body 44, it is possible to solve a trouble that the second ferrule 41B moves in a direction of an arrow Z1 by vibration, and the distal end face of the illumination fiber 14 moves in the same direction.

In addition, by the contact face 42f of the diameter-increased portion 42 coming into contact with the proximal end face 44r of the holding body 44, a trouble is reduced that, when vibration is propagated from the diameter-increased portion 42 to the holding body 44, the vibration propagated as shown by broken-line arrows Y6A in FIG. 6A is reflected by an interface with the flange portion 41Bf arranged in the holding body 44, and, thereby, the holding body 44 is vibrated.

Note that a height of projection of the flange portion 41Bf is set to be a same size as the height of the projection of the diameter-increased portion 42 or lower. Further, it is also possible to provide a circumferential recess groove on the second ferrule 41B and arrange the recess groove in the holding body 44.

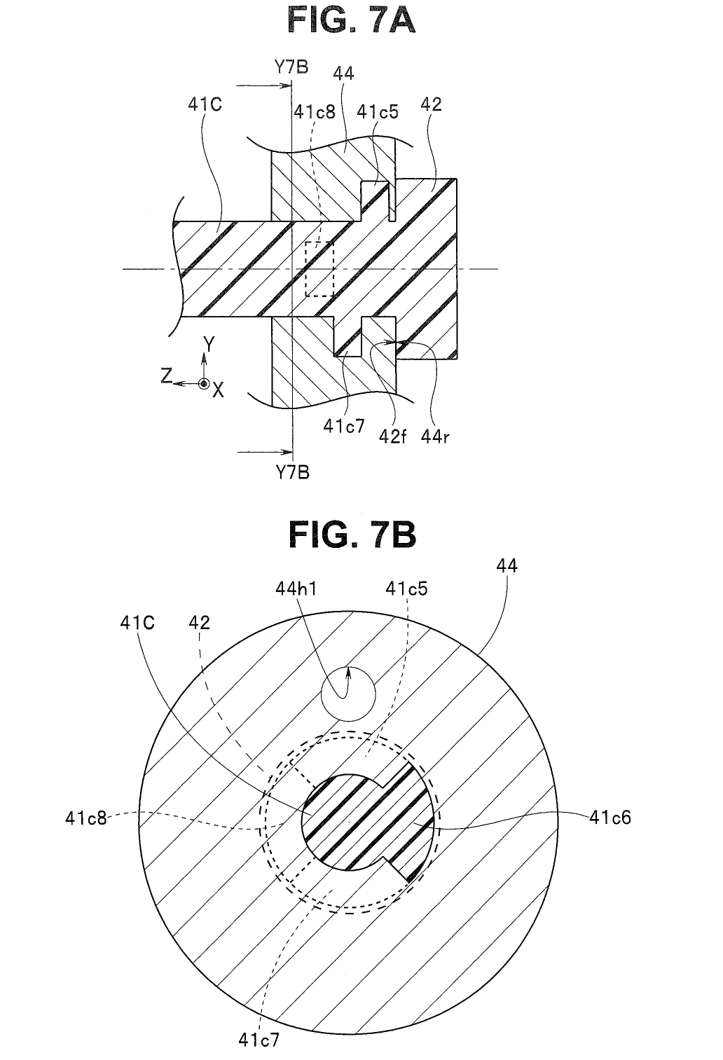

A third ferrule 41C shown in FIGS. 7A and 7B has fan-shaped projection portions 41c5, 41c6, 41c7 and 41c8 projecting outward in a circumferential direction. The fan-shaped projection portions 41c5, 41c6, 41c7 and 41c8 are provided at intervals in the Z direction as shown in FIG. 7A and provided at intervals in a circumferential direction, for example, with a center angle of 90 degrees as shown in FIG. 7B.

Note that, in the present embodiment, the fan-shaped projection portions 41c5, 41c6, 41c7 and 41c8 are separated at equal intervals in the Z direction and separated at intervals of 90 degrees in the circumferential direction.

The fan-shaped projection portions 41c5, 41c6, 41c7 and 41c8 are arranged in the holding body 44 to prevent the ferrule 41C from moving in the Z direction and rotating in the circumferential direction.

By arranging the fan-shaped projection portions 41c5, 41c6, 41c7 and 41c8 in the holding body 44 at intervals of 90° in the circumferential direction as shown in FIG. 7B, the fan-shaped projection portions 41c5, 41c6, 41c7 and 41c8 can be provided without a gap, like the flange portion 41Bf described above.

Thereby, it is possible to block vibration transmitted from the diameter-increased portion 42 to the holding body 44 from traveling in an axial direction by the respective fan-shaped projection portions 41c5, 41c6, 41c7 and 41c8 as described above.

As a result, operation and effects similar to those of the flange portion 41Bf described above can be obtained.

According to the configuration, a trouble that the ferrule 41C rotatingly moves in the circumferential direction by vibration, a trouble that the ferrule 41C moves in the axial direction and the trouble that vibration is transmitted from the diameter-increased portion 42 to the holding body 44 are reduced.

Note that operation and effects similar to those of the embodiment described above may be obtained by arranging a ferrule provided with a spiral-shaped projection portion on an outer circumferential face in the holding body 44 instead of arranging the fan-shaped projection portions 41c5, 41c6, 41c7 and 41c8 at equal intervals in the Z direction. Further, the number of fan-shaped projection portions is not limited to four. In a case of two fan-shaped projection portions, the center angle is set to 180 degrees, and, in a case of three, the center angle is set to 120 degrees.

The invention described in the embodiment described above is not limited to the embodiment and the modifications. In addition, at an implementation phase, it is possible to make various modifications within a range not departing from the spirit of the invention.

According to the present invention, it is possible to realize a scanning-type endoscope which prevents vibration from being transmitted to the scanner driving portion from outside and prevents vibration from being transmitted to a scanner driving portion holding portion and a lens frame from the scanner driving portion to obtain a preferable observation target image.

What is claimed is:

1. A scanning-type endoscope comprising:
a scanning portion comprising a light guiding portion configured to guide illuminating light emitted from a light source portion and emit the illuminating light from a distal end, and an actuator configured to oscillate the distal end of the light guiding portion in order to perform scanning on an observation target with the illuminating light;
a cylindrical member including a space containing the scanning portion and provided along the light guiding portion; and
a holding portion provided on the cylindrical member and configured to hold the scanning portion at a predetermined position on a plane perpendicular to a longitudinal direction of the cylindrical member and configured so that the scanning portion is slidable in the longitudinal direction.

2. The scanning-type endoscope according to claim 1, wherein
the holding portion comprises a groove portion or a projecting portion provided along the longitudinal direction; and
the scanning portion comprises a projecting portion or a groove portion fittedly arranged in the groove portion or the projecting portion provided on the holding portion.

3. The scanning-type endoscope according to claim 2, wherein
on the holding portion, the groove portion or the projection portion is provided in plurality at intervals in the longitudinal direction; and
on the scanning portion, the projecting portion or the groove portion that the scanning portion comprises is provided in plurality at intervals at positions corresponding to positions of the groove portion or the projecting portion that the holding portion comprises.

4. The scanning-type endoscope according to claim 1, further comprising an optical member configured to condense return light from the observation target.

5. The scanning-type endoscope according to claim 1, wherein
the scanning portion is provided around the light guiding portion and comprises a ferrule with the actuator arranged on an outer circumferential of the ferrule; and
the ferrule is fitted in the holding portion and is slidable in the state of being fitted in the holding portion.
